(19) United States
(12) Patent Application Publication
(10) Pub. No.: US 2003/0082654 A1
(43) Pub. Date: May 1, 2003

(54) Novel cancer marker and uses therefor in the diagnosis of cancer

(76) Inventors: Christopher Richard Parish, Campbell (AU); Vivian Mae Cabalda-Crane, Evatt (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/212,856

(22) Filed: Aug. 5, 2002

Related U.S. Application Data
(60) Provisional application No. 60/309,907, filed on Aug. 3, 2001.

(51) Int. Cl. G01N 33/574; C07K 14/47; C07K 7/08

(52) U.S. Cl. 435/7.23; 530/324

(57) ABSTRACT

Provided are novel cancer markers for the diagnosis of cancer in humans and non-human mammalian subjects, specifically a cancer marker comprising a negatively-charged molecule with a mass/charge (m/z) ratio of about 991. The cancer marker of the invention may be used to determine the presence of one or more cancerous cells or tumors in a biological sample by assaying the sample for a reduced level of said cancer marker.

This application claims priority of United States Provisional Application No. 60/309,907, filed Aug. 3, 2001, herein incorporated by reference in its entirety.

### FIELD OF THE INVENTION

This invention relates to a novel cancer marker for the diagnosis of cancer in humans and non-human mammalian subjects, specifically a cancer marker comprising a negatively-charged molecule with a mass/charge (m/z) ratio of about 991. The cancer marker described herein may be used to determine the presence of one or more cancerous cells or tumors in a biological sample from a subject, such as, for example, a bodily fluid, by assaying a biological sample from said subject for a reduced level of said cancer marker.

### BACKGROUND OF THE INVENTION

In spite of numerous advances in medical research, cancer remains a major cause of death worldwide, and there is a need for rapid and simple methods for the early diagnosis of cancer, to facilitate appropriate remedial action by surgical resection, radiotherapy, chemotherapy, or other known treatment methods. The availability of good diagnostic methods for cancer is also important to assess patient responses to treatment, or to assess recurrence due to re-growth at the original site or metastases.

The characterization of cancer markers, such as, for example, oncogene products, growth factors and growth factor receptors, angiogenic factors, proteases, adhesion factors and tumor suppressor gene products, etc, can provide important information concerning the risk, presence, status or future behavior of cancer in a human or non-human mammalian subject. Determining the presence or level of expression or activity of one or more cancer markers can assist the differential diagnosis of patients with uncertain clinical abnormalities, for example by distinguishing malignant from benign abnormalities. Furthermore, in patients presenting with established malignancy, cancer markers can be useful to predict the risk of future relapse, or the likelihood of response in a particular patient to a selected therapeutic course. Even more specific information can be obtained by analyzing highly specific cancer markers, or combinations of markers, which may predict responsiveness of a patient to specific drugs or treatment options.

It is well known that aberrant glycosylation is a common feature for most cancer types, and drastic changes to serine/threonine-linked glycan (i.e. O-glycan) levels may occur in cancer patients. “O-glycan” is a glycoprotein wherein N-acetylgalactosamine is added to serine and/or threonine residues of nascent protein. Cancer patients may, for example, have a reduced level of common O-glycan core structures, enhanced levels of sialylated glycan or ganglioside, or decreased modification to sialic acid. The synthesis of specific peptide moieties of O-glycans may also be altered in cancer patients, thereby modifying O-glycan levels, since the peptide moieties of glycoproteins in part direct the synthesis of O-glycans. Alternatively, sialyltransferase activities may be enhanced in cancer patients, thereby producing hypersialylated O-glycans.

Generally, tumor-specific antigens are high molecular weight or high molecular mass molecules (>10,000 Da) that are either expressed specifically on a cancer cell or expressed at elevated levels on cancer cells compared to normal cells. However, there are low molecular weight (<10,000 Da) tumor-specific antigens which are often glycolipids, more particularly sphingolipids, that comprise polylactosamine structures. A “glycolipid” is simply a lipid or fatty acid molecule having one or more carbohydrate moieties.

“Sphingolipids” are lipids comprising a fatty acid residue, a polar head group, and sphingosine (4-sphingenine) or a related base, including ceramide, and its derivatives, sphingomyelin (i.e. ceramide that comprises a phosphocholine moiety on the hydroxyl group), or the glycosphingolipids (i.e. ceramide comprising a carbohydrate moiety on the hydroxyl group), including a ganglioside.

A “ganglioside” is a glycosphingolipid that contains sialic acid (i.e. a glycolipid wherein a fatty acid-substituted sphingosine is linked to an oligosaccharide that comprises D-glucose, D-galactose, N-acetylgalactosamine and/or N-acetylneuraminic acid) and which is expressed in the majority of mammalian cell membranes. Gangliosides are mono-, di-, tri, or poly- sialogangliosides, depending upon the extent of glycosylation with sialic acid. In accordance with standard nomenclature, the terms “GMn”, “GDn”, “GTn”, are used, wherein “G” indicates a ganglioside; “M” indicates a monosialyl ganglioside, “D” indicates a disialyl ganglioside, and “T” indicates a trisialyl ganglioside; and wherein “n” is a numeric indicator having a value of at least 1, or an alphanumeric indicator having a value of at least 1 a (e.g. 1a, 1b, 1c, etc), indicating the binding pattern observed for the molecule [Lehninger, In: Biochemistry, pp. 294-296 (Worth Publishers, 1981); Wiegandt, In: Glycolipids: New Comprehensive Biochemistry, pp. 199-260 (Neuberger et al., ed., Elsevier, 1985)].

Polylactosamines are usually classified into two categories according to their polylactosamine unit structure, in particular Type 1 polylactosamines comprising galactosyl-(∃1-3,) N-acetylglucosamine, or alternatively, Type 2 polylactosamines comprising galactosyl (∃1-4) N-acetylglucosamine.

Gangliosides, such as, for example, GM2 (Livingston et al., Proc. Natl. Acad. Sci. USA 84, 2911-2915, 1987), GD2 (Schulz, et al., Cancer Res. 44, 5914-5920, 1984), or GD3 (Cheresh et al., Proc. Natl. Acad. Sci. USA 81, 5767-5771, 1984; Reisfeld et al., In: Immunity to Cancer (M. S. Mitchell, Ed), pp 69-84, 1985), have been identified as prominent cell surface constituents of various tumors of neuroectodermal origin, such as, for example, malignant melanoma, neuroblastoma, glioma, soft tissue sarcoma and small cell carcinoma of the lung. These gangliosides are absent, or present at only low levels, in most normal tissues. The role of gangliosides as tumor-specific antigens is also discussed, for example, by Ritter and Livingston, et al., Sem. Cane. Biol. 2, 401-409, 1991; Chatterjee et al., U.S. Pat. No. 5,977,316 issued Nov. 2, 1999; Hakomori Cancer Res. 45, 2405-2414, 1985; Miraldi In:Seminars in Nuclear Medicine XIX, 282-294, 1989; and Hamilton et al, Int. J. Cancer 53, 1-8, 1993.

A common tumor-associated antigen found in major cancers are gangliosides that comprise the Type 2 chain polylactosamine structure, or alternatively, the fucosylated form. For example, the gangliosides sialyl-Lewis A and sialyl-Lewis X are involved in the adhesion of cancer cells to vascular endothelial cells, and contribute to the hematogenous metastasis of cancer. Sialyl-Lewis A is frequently expressed in cancers of the colon, pancreas and biliary tract, whilst sialyl-Lewis X is commonly expressed in cancers of the breast, lung, liver and ovary. The degree of expression of the carbohydrate ligands of sialyl-Lewis A or sialyl-Lewis X at the surface of cancer cells is well correlated with the frequency of hematogenous metastasis and prognostic outcome of patients with cancers.

On the other hand, gangliosides comprising the Type 1 polylactosamine structure, such as, for example, 2-3 sialyl Lewis A, are abundant in normal cells and tissues, and are also cancer-associated. Levery et al (U.S. Pat. No. 6,083,929 issued Jul. 2, 2000) teach that extended forms of lacto-series Type 1 chain, with or without sialyl and/or fucosyl residues, are present in cancer tissues. Levery et al (ibid.) showed that an isoform isolated from the glycolipid fraction of the colon adenocarcinoma cell line Colo205 comprised the following glycosphingolipid units: homodimeric LewisA, heterodimeric LewisB-LewisA, and extended sialyl LewisA-LewisA, the latter of which is suggested as a tumor-associated glycosphingolipid and potential tumor marker.

However, despite the progress in identifying sialylated antigens for the detection of cancer, there remains a clear need for cancer markers to assist in the diagnosis of cancers, and the detection of specific cancer types. In particular, notwithstanding the perturbation of glycosylation observed in cancer, there are few, if any, known cancer markers that are not necessarily sialylated compounds or O-linked glycoproteins, and/or are not tumor-specific antigens.

A preferred characteristic of a cancer marker is that it is readily amenable to detection using rapid or high throughput analytical methods, such as, for example, mass spectrometry, or high pressure liquid chromatography (HPLC)-mass spectrometry.

Furthermore, a suitable cancer marker should be amenable to detection in a bodily fluid (e.g. blood, serum, urine, mucus, saliva, sweat, tears or other fluid secretion), thereby facilitating the use of non-invasive assays for routine testing.

### SUMMARY OF THE INVENTION

In work leading up to the present invention, the inventors sought to identify both high and low molecular weight/mass cancer markers in the bodily fluids of humans and non-human mammalian subjects, and to develop related high throughput diagnostic methods for the detection of malignancies associated with a reduced level of such cancer markers in a bodily fluid, wherein such diagnostics did not depend upon the isolation of a molecular probe, such as, for example, an antibody or nucleic acid probe, and/or did not require a time-consuming binding step using such a molecular probe.

Accordingly the first aspect of the present invention provides a cancer marker comprising a negatively-charged molecule with a m/z ratio of about 991 that is present at a reduced level in a subject having a cancer compared to a healthy subject, or a derivative of said negatively-charged molecule.

A second aspect of the present invention provides a method of diagnosing or detecting cancer in a human or non-human mammalian subject comprising:

(i) determining the level of a cancer marker in a test sample from a subject suspected of having cancer, said cancer marker comprising a negatively-charged molecule having a m/z ratio of about 991 or a derivative thereof; and

(ii) comparing the level of the cancer marker or derivative at (i) to the level of the cancer marker or derivative in a control sample from a healthy subject, or the level established for a healthy subject, wherein a reduced level of said cancer marker or derivative relative to the level in the healthy subject, or the level established for a healthy subject, is indicative of cancer.

A third aspect of the present invention provides a method of diagnosing or detecting cancer in a human or non-human mammalian subject comprising:

(i) determining the level of a cancer marker in a test sample from a subject suspected of having cancer, said cancer marker comprising a negatively-charged molecule having a m/z ratio of about 991 or a derivative thereof; and

(ii) comparing the level of the cancer marker or derivative at (i) to the level of an internal standard added to the test sample, wherein a reduced level of said cancer marker or derivative relative to the level of the internal standard is indicative of cancer.

A fourth aspect of the present invention provides a method of diagnosing or detecting cancer in a human or non-human mammalian subject comprising determining the level of a cancer marker in a test sample from a subject suspected of having cancer, said cancer marker comprising a negatively-charged molecule having a m/z ratio of about 991 or a derivative thereof; relative to the level of another marker in the same test sample, wherein a change in the ratio of the cancer marker to the another marker is indicative of cancer.

A fifth aspect of the present invention provides a method of monitoring cancer treatment in a human or non-human mammalian subject comprising:

(i) determining the level of a cancer marker in a test sample from a subject being treated for cancer, said cancer marker comprising a negatively-charged molecule having a m/z ratio of about 991 or a derivative thereof; and

(ii) comparing the level of the cancer marker or derivative at (i) to the level of the cancer marker or derivative in a control sample from a healthy subject, the level established for a healthy subject, wherein an increased level is indicative of successful treatment.

A sixth aspect of the present invention provides a method of diagnosing recurrence of cancer following successful treatment in a human or non-human mammalian subject comprising:

(i) determining the level of a cancer marker in a test sample from a subject treated for cancer, said cancer marker comprising a negatively-charged molecule having a m/z ratio of about 991 or a derivative thereof; and

(ii) comparing the level of the cancer marker or derivative at (i) to the level of the cancer marker or derivative in a control sample from a healthy subject, the level established for a healthy subject or the level in a sample from the subject following successfully treated for cancer, wherein a reduced level is indicative of recurrence of cancer.

### Definitions

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps, features, compositions and compounds.

The present invention is not to be limited in scope by the specific embodiments described herein, which are intended for the purposes of exemplification only. Functionally equivalent products, compositions and methods are clearly within the scope of the invention, as described herein.

The reference to any prior art document(s) in this specification is made merely for the purposes of further describing the instant invention and is not to be taken as an indication or admission that said document(s) forms part of the common general knowledge of a skilled person in Australia or elsewhere.

As used herein the words “from” or “of”, and the term “derived from” shall be taken to indicate that a specified product, in particular a molecule such as, for example, a polypeptide, protein, gene or nucleic acid molecule, antibody molecule, Ig fraction, or other molecule, or a biological sample comprising said molecule, may be obtained from a particular source, organism, tissue, organ or cell, albeit not necessarily directly from that source, organism, tissue, organ or cell.

As used herein, “cancer” shall be taken to mean any one or more of a wide range of benign or malignant tumors, including those that are capable of invasive growth and metastasise through a human or non-human mammalian body or a part thereof, such as, for example, via the lymphatic system and/or the blood stream. As used herein, the term “tumor” includes both benign and malignant tumors or solid growths, notwithstanding that the present invention is particularly directed to the diagnosis or detection of malignant tumors and solid cancers. Typical cancers include but are not limited to carcinomas, lymphomas, or sarcomas, such as, for example, ovarian cancer, colon cancer, breast cancer, pancreatic cancer, lung cancer, prostate cancer, urinary tract cancer, uterine cancer, acute lymphatic leukemia, Hodgkin's disease, melanoma, neuroblastoma, glioma, and soft tissue sarcoma.

In the context of the present invention as described herein and defined by the claims, the term “cancer marker” shall be taken to mean any molecule that is detectable in a biological sample from a human or non-human mammalian subject, such as, for example, a bodily fluid (blood, urine, mucus, saliva, sweat, tear or other fluid secretion) and is indicative of cancer in the subject, specifically a molecule whose level is reduced in a bodily fluid of a subject having cancer compared to its level in a bodily fluid of a healthy subject. The term “cancer marker” shall also be taken to include a molecule that is expressed by or on a normal cell but not on a cancer cell or whose expression is reduced by or on a cancer cells compared to a normal cell.

The term “negatively-charged molecule” is used interchangeably in the context of the present invention with the terms “negatively-charged carbohydrate-containing molecule” or “carbohydrate-containing molecule”, to refer to the cancer marker of the present invention having m/z ratio of about 991, whether or not the marker in fact comprises carbohydrate as part of the molecule. The terms also include in their scope a derivative of the molecule such as, for example, a derivative that comprises phosphate or sulfate.

When the molecule comprises carbohydrate, it preferably comprises a monosaccharide, disaccharide, or oligosaccharide (i.e. at least three and no more than about nine monosaccharide units).

### BRIEF DESCRIPTION OF THE DRAWINGS

FIG. 1A is a graphical representation of a Matrix-Assisted Laser Desorption Ionization-Time of Flight (MALDI-TOF) mass spectrometer profile of a fraction of serum from untreated rats that is eluted from a C₁₈ solid phase Seppak cartridge using water as the eluant. The x-axis indicates mass to charge ratio (m/z), and the ordinate refers to the relative abundance of each molecular species as a percentage of the abundance of the most abundant species. Numbers at the top of each peak refer to m/z ratio of that peak. The arrow indicates the position of a prominent negative ion (m/z 991) that is reduced in subjects suffering from adenocarcinoma (FIG. 1B).

FIG. 1B is a graphical representation of a Matrix-Assisted Laser Desorption Ionization-Time of Flight (MALDI-TOF) mass spectrometer profile of a fraction of serum from tumor-bearing rats that is eluted from a C₁₈ solid phase Seppak cartridge using water as the eluant. The tumor-bearing rats were assayed 13 days after subcutaneous injection (10⁶ cells/rat) with the highly malignant and metastatic rat mammary adenocarcinoma 13762 MAT. The x-axis indicates mass to charge ratio (m/z), and the ordinate refers to the relative abundance of each molecular species as a percentage of the abundance of the most abundant species. Numbers at the top of each peak refer to the m/z ratio of that peak. The arrow indicates the position of the negative ion (m/z 991) that is prominent in the spectra from untreated rats (FIG. 1A).

FIG. 2A is a graphical representation of a Matrix-Assisted Laser Desorption Ionization-Time of Flight (MALDI-TOF) mass spectrometer profile of a fraction of serum from normal, untreated, mice that is eluted from a C₁₈ solid phase Seppak cartridge using methanol as the eluant. The x-axis indicates mass to charge ratio (m/z), and the ordinate refers to the relative abundance of each molecular species as a percentage of the abundance of the most abundant species. Numbers at the top of each peak refer to the m/z ratio of that peak. The arrow indicates the position of a prominent negative ion (m/z 991) that is reduced in tumor-bearing mice (FIG. 2B).

FIG. 2B is a graphical representation of a Matrix-Assisted Laser Desorption Ionization-Time of Flight (MALDI-TOF) mass spectrometer profile of a fraction of serum from tumor-bearing mice that is eluted from a C₁₈ solid phase Seppak cartridge using methanol as the eluant. Tumor-bearing mice were assayed at 15 days after subcutaneous injection (10⁶ cells/mouse) with the highly malignant and metastatic B16F1 melanoma. The x-axis indicates mass to charge ratio (m/z), and the ordinate refers to the relative abundance of each molecular species as a percentage of the abundance of the most abundant species. Numbers at the top of each peak refer to the m/z ratio of that peak. The arrow indicates the position of the negative ion (m/z 991) that is prominent in the spectra from untreated mice (FIG. 2A).

FIG. 3A is a graphical representation of a Matrix-Assisted Laser Desorption Ionization-Time of Flight (MALDI-TOF) mass spectrometer profile of a fraction of serum from normal, untreated, humans that is eluted from a C₁₈ solid phase Seppak cartridge using water as the eluant. The x-axis indicates mass to charge ratio (m/z), and the ordinate refers to the relative abundance of each molecular species as a percentage of the abundance of the most abundant species. Numbers at the top of each peak refer to the m/z ratio of that peak. The arrow indicates the position of a prominent negative ion (m/z 991) that is reduced in colon cancer patients (FIG. 3B).

FIG. 3B is a graphical representation of a Matrix-Assisted Laser Desorption Ionization-Time of Flight (MALDI-TOF) mass spectrometer profile of a fraction of the plasma of colon cancer patients that is eluted from a C₁₈ solid phase Seppak cartridge using water as the eluant. The x-axis indicates mass to charge ratio (m/z), and the ordinate refers to the relative abundance of each molecular species as a percentage of the abundance of the most abundant species. Numbers at the top of each peak refer to the m/z ratio of that peak. The arrow indicates the position of the negative ion (m/z 991) that is prominent in the spectra from normal, untreated, human subjects (FIG. 3A).

FIG. 4A is a graphical representation of a Matrix-Assisted Laser Desorption Ionization-Time of Flight (MALDI-TOF) mass spectrometer profile of fragments of the negative ion (m/z 991) from normal, untreated mouse serum (FIG. 1A), obtained using Matrix-Assisted Laser Desorption Ionization-Time of Flight (MALDI-TOF) mass spectrometry-based post source decay fragmentation. The x-axis indicates the mass/charge ratio (m/z), and the ordinate indicates the abundance of each fragment. Numbers at the top of each peak refer to the m/z ratio of that peak. Major fragments having m/z ratios, from right to left in the figure, of 241, 644, 705, 749, and 947. The position of the intact m/z 991 negative ion species is also indicated at the far right of the spectrum. The m/z 241 ion fragment is consistent with a hexose phosphate moiety, such as inositol phosphate, or hexose sulfate.

FIG. 4B is a graphical representation of a Matrix-Assisted Laser Desorption Ionization-Time of Flight (MALDI-TOF) mass spectrometer profile of fragments of the negative ion (m/z 991) from normal, untreated rat serum (FIG. 2A), obtained using Matrix-Assisted Laser Desorption Ionization-Time of Flight (MALDI-TOF) mass spectrometry-based post source decay fragmentation. The x-axis indicates mass/charge ratio (m/z), and the ordinate indicates the abundance of each fragment. Numbers at the top of each peak refer to the m/z ratio of that peak. Major fragments having m/z ratios, from right to left in the figure, of 241, 644, 705, 749, and 947. The position of the intact m/z 991 negative ion species is also indicated at the far right of the spectrum. The m/z 241 ion fragment is consistent with a hexose phosphate moiety, such as inositol phosphate, or hexose sulfate. The high background is most likely a consequence of their being a small amount of the intact m/z 991 negative ion in the sample.

FIG. 4C is a graphical representation of a Matrix-Assisted Laser Desorption Ionization-Time of Flight (MALDI-TOF) mass spectrometer profile of fragments of the negative ion (m/z 991) from the serum of a healthy human (FIG. 3A), obtained using Matrix-Assisted Laser Desorption Ionization-Time of Flight (MALDI-TOF) mass spectrometry-based post source decay fragmentation. The x-axis indicates mass/charge ratio (m/z), and the ordinate indicates the abundance of each fragment. Numbers at the top of each peak refer to the m/z ratio of that peak. Major fragments having m/z ratios, from right to left in the figure, of 241, 644, 705, 749, and 947. The position of the intact m/z 991 negative ion species is also indicated at the far right of the spectrum. The m/z 241 ion fragment is consistent with a hexose phosphate moiety, such as inositol phosphate, or hexose sulfate.

### DETAILED DESCRIPTION OF THE PREFERRED EMBODIMENTS

One aspect of the present invention provides a cancer marker comprising a negatively-charged molecule with a m/z ratio of about 991 that is present at a reduced level in a subject having a cancer compared to a healthy subject or a derivative of said negatively-charged molecule.

Preferably, the negatively-charged molecule of the invention is provided in isolated form. By “isolated” means substantially free of conspecific glycolipids, disaccharides, monosaccharides, or oligosaccharides, such as, for example, determined by mass spectrometry under the conditions defined herein. By virtue of the high resolution of MALDI-TOF MS, it will be understood by the skilled person that the mass spectrometry profile of post-source ionization fragments of the m/z 991 ionic species corresponds to a “fingerprint” of that molecule.

Preferably, the carbohydrate moiety, when present, comprises hexosephosphate or hexose sulfate. In this respect, post-source decay fragmentation data reveal that the isolated negatively-charged molecule produces a fragment having a m/z ratio, as estimated by MALDI-TOF MS, of about 241, that is characteristic of hexose-phosphate, such as, for example, phosphatidylinositol (i.e. inositol-1,2 cyclic phosphate). Even more preferably, the carbohydrate moiety comprises glycosylphosphatidylinositol (GPI). Still more preferably, the carbohydrate-containing molecule comprises a disaccharide or oligosaccharide moiety comprising at least one hexose phosphate, phosphatidylinositol, or GPI unit.

Also in the present context, the term “negatively-charged carbohydrate-containing molecule”, or “negatively-charged molecule” or its interchangeable terms as set out above, shall be taken to mean that the molecule is sufficiently hydrophilic that it does not bind strongly to a hydrophobic matrix, in particular a C-18 matrix, and preferably comprises one or more phosphorus or sulfate atoms. In this respect, ionization of the cancer marker of the invention using mass spectrometry, in particular, Matrix-Assisted Laser Desorption Ionization-Time of Flight Mass Spectrometry (MALDI-TOF MS) indicates that the isolated cancer marker of the invention is a negatively-charged ion. Accordingly, the term “negatively charged carbohydrate-containing molecule” includes a phospholipid, phosphoglyceride, phosphate-containing N-linked glycoprotein, phosphate-containing O-linked glycoprotein, phosphatidylinositol-containing lipid or protein, or a glycosylphosphatidylinositol (GPI)-containing lipid or protein.

The cancer marker described herein has been analyzed according to a selection of its properties, and it is proposed that a moiety of said cancer marker may be linked in situ to other functional groups. For example, where the moiety is a monosaccharide, disaccharide or oligosaccharide, the moiety can be O-linked or N-linked in situ to a proteinaceous moiety (e.g. an amino acid, a peptide, or polypeptide) to form a glycopeptide/glycoprotein, or alternatively or in addition, it may be linked in situ to a lipid moiety, such as, for example, a fatty acid (palmitic acid and/or oleic acid and/or myristic acid and/or arachidonic acid, amongst others); triacylglycerol; a phospholipid; phosphoglyceride (e.g. phosphatidyl choline, phosphatidyl serine, phosphatidyl inositol, phosphatidyl glycerol, or phosphatidyl ethanolamine, amongst others); sphingolipid; sphingosine; or cholesterol hormone. All such variants may be used as a cancer marker within the context described herein. Accordingly, the present invention clearly encompasses peptide or lipid variants of the moiety, the only requirement being that such variants comprise the m/z 991 ionic species.

Still more preferably, the cancer marker comprises a glycolipid, and, even more preferably, a glycolipid comprising phosphatidylinositol and one or more fatty acids selected from the group consisting of myristic acid, palmitic acid, and oleic acid. The structure of the lipid moiety of the cancer marker described herein is elucidated using any one or more of several techniques known to those skilled in the art, without under experimentation, in particular Fast Atom Bombardment (FAB), Collisionally Activated Dissocation (CAD), Tandem Mass Spectrometry, essentially as described by Ladisch et al., J. Biol. Chem. 264, 12097-12105, 1989, or P-NMR techniques, amongst others.

This embodiment of the invention clearly extends to a derivative of said glycolipid, such as, for example, a derivative that comprises one or more fluorescent ligands, enzyme ligands, radioactive ligands, peptide ligands (e.g. FLAG), or antibody ligands, covalently linked to the m/z 991 ion to facilitate its detection.

In a particularly preferred embodiment of the present invention, the cancer marker comprises dimyristoyl-phosphatidylinositol (i.e. dimyristoyl-PI), optionally acylated with an additional fatty acid, such as, for example, palmitic acid or oleic acid. This characterization of the cancer marker of the present invention is consistent with both the m/z 991 ion value for the intact molecule during MALDI-TOF MS, and the appearance of a m/z 241 peak during post-source fragmentation analysis. This embodiment of the invention clearly extends to a derivative of said glycolipid, such as, for example, a derivative that comprises one or more fluorescent ligands, enzyme ligands, radioactive ligands, peptide ligands (e.g. FLAG), or antibody ligands, covalently linked to the glycolipid to facilitate its detection.

The molecular mass and/or mass charge ratio or other physical property of the molecule of the invention can be determined by any art-recognized method, including gel filtration, gel electrophoresis, capillary electrophoresis, mass spectrometry, HPLC, FPLC, or by predicting the molecular mass of the compound from compositional or structural data. Preferably, the mass charge ratio is determined by mass spectrometry, including MALDI-TOF MS, tandem MS, electrospray MS, etc.

Reference herein to a mass/charge ratio or m/z ratio as being “about” a specified value shall be understood by those skilled in the art to include an acceptable variation without its further definition. Preferably, m/z ratio estimates as determined by mass spectrometry of samples that are recited herein include an acceptable error of m/z±5, more preferably m/z±4, even more preferably m/z±3, still more preferably m/z±2, and even still more preferably m/z±1. Accordingly, it shall be understood that an estimated m/z ratio of about 991 includes a m/z ratio in the range of 986-996, preferably in the range of 987-995, more preferably in the range of 988-994, even more preferably in the range of 989-993, and still more preferably in the range of 990-992, or even 991.

As used herein, a “derivative” of the cancer marker of the present invention shall be taken to mean any molecule produced from the parent molecule with an m/z ratio of about 991 described herein.

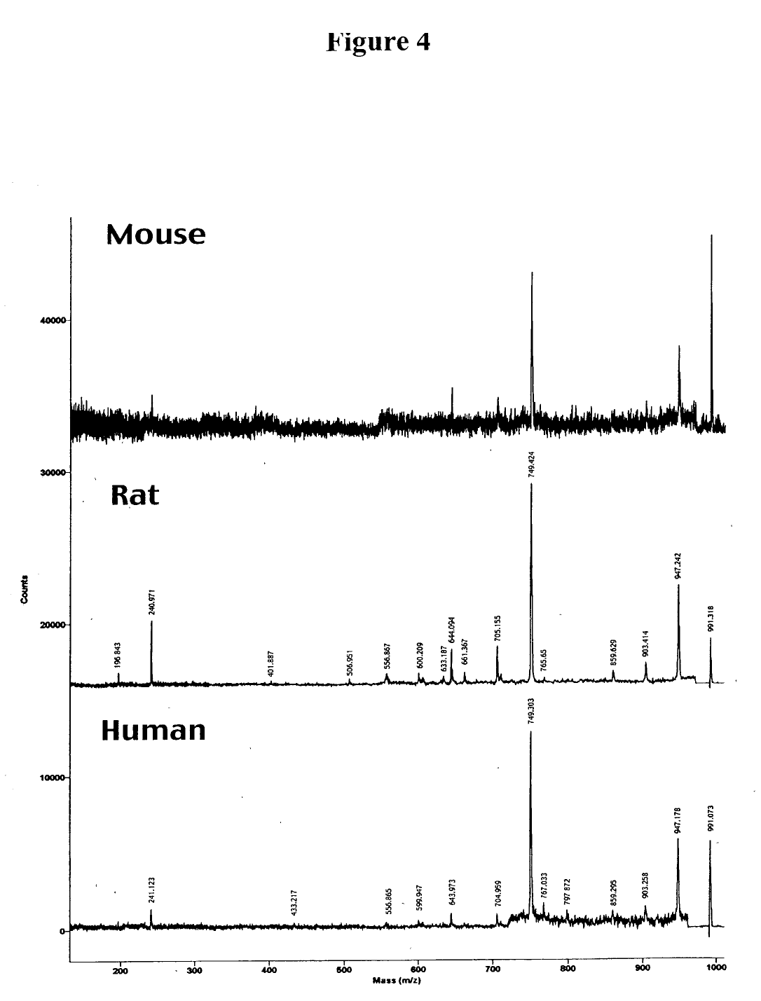

The derivatives of the present invention thus include any and all fragments of the molecule of the invention and their use as cancer markers, the only requirement being that the fragments retain the specificity of the parent molecule with respect to cancer detection assays. As will be apparent from the disclosure provided herein (particularly in FIGS. 4A, 4B, and 4C), the molecule of the invention produces a specific “fingerprint” on post-source ionization, with fragments of m/z about 241, about 644, about 705, about 749, and about 947 being generated. A high background of monosaccharides or inositol phosphate in a sample may result in masking of one or more of the characteristic fragment peaks. In such cases, those skilled in the art will be aware that the presence of two fragments, preferably three fragments, more preferably four fragments, and more preferably all five fragments, can be used as a cancer marker having the specificity of the parent molecule.

Preferred derivatives of the molecule include, for example, fragments of the carbohydrate moiety of a carbohydrate-containing form of said molecule that are produced by standard means known to those skilled in glycobiology. As such analyses frequently depend upon the chemical modification to facilitate their detection, the present invention also extends to include any chemically-modified fragment of the m/z 991 cancer marker of the invention produced by permethylation, periodate oxidation, NaBH₄ reduction, reductive amination, (e.g. using 2-aminopyridine), or by incubation with perfluorobenzylaminobenzoate or alkylaminobenzoate, amongst others. Derivatives further include any carbohydrate-containing or non-carbohydrate-containing molecule produced by a combination of the foregoing processes.

Those skilled in the art will be aware of several well-known means for determining the precise structure of a carbohydrate moiety of the subject cancer marker when such moieties are present. In some cases, derivatives of such carbohydrate moieties can be produced through any of a variety of well-known techniques. Moreover, a variety of determination mechanisms (e.g. enzyme digestion or fingerprinting techniques, mass spectrometry, tandem mass spectrometry, high pressure liquid chromatography (HPLC)-mass spectrometry, molecular modeling, lectin affinity chromatography (especially in conjunction with high performance liquid affinity chromatography, hereinafter “lectin-HPLAC”), reverse phase methods, size exclusion, etc.) can be used to identify the derivativized and underivatized carbohydrate moieties can be used.

Overall carbohydrate composition, when carbohydrates are present, to provide the number and type of monosaccharide residues, or determine the presence of N-acetylgalactosamine or O-glycan, is determined, for example, by acid hydrolysis, or methanolysis, to release the monosaccharides as reducing sugars or methyl glycosides, respectively. Gas chromatography (GC) and/or liquid chromatography, under low or high pressure, is then used to resolve, and quantify, released monosaccharides. For GC, and optionally for liquid chromatography, monosaccharides are generally derivatized, such as, for example, by permethylation. High pH anion-exchange chromatography with pulsed amperometric detection (HPAEC/PAD), as described essentially by Hardy, Methods Enzymol. 179, 76-82, 1989 is also used.

For elucidating the carbohydrate moiety of a glycoprotein, it is necessary to release smaller carbohydrate units (monosaccharides, disaccharides, oligosaccharides), such as, for example, using chemical and/or enzymatic methods. Enzyme digestion methods include incubation with an effective amount of a peptide N-glycosidase F (EC 3.2.2.18,) or other endoglycosidase or glycoamidase (see Takahashi, N. and Muramatsu, T., Eds. (1992) In: CRC Handbook of Endoglycosidases and Glycoamidases, CRC Press, Inc., Boca Raton, Fla.), or an endo-beta-N-acetylglucosidase (EC 3.2.1.96) or glycosidase, such as, for example, Endo H or Endo F (see Maley, F. et al., Anal. Biochem. 180, 195-204, 1989), to effect release. Chemical methods include incubation for a time and under conditions sufficient to effect carbohydrate release, with anhydrous hydrazine, or a strong alkali in combination with a reducing agent, optionally in combination with NaBH₄.

Structure of the released carbohydrate from a carbohydrate-containing form of the molecule can be determined, for example, by sequential digestion using exoglycosidase, regiospecific chemical degradation, methylation analysis (GC-MS), FAB-MS, and/or high-field proton and multidimensional NMR methods. To facilitate resolution of the carbohydrate-containing fragments generated, they are derivatized with a chromophore or fluorophore, or radiochemical. Pulsed amperometry (PAD) can also be used to facilitate the resolution of non-derivatized carbohydrates.

Spectrometric techniques, such as, for example, mass spectrometry, high pressure liquid chromatography (HPLC), or combination techniques, such as, for example, tandem mass spectrometry, high pressure liquid chromatography (HPLC)-mass spectrometry, are preferred for the separation of complex mixtures of carbohydrate-containing and non-carbohydrate-containing molecules. Excellent reviews are available in the literature (see, for example Honda, Anal. Biochem. 140, 1-47, 1984; Townsend. (1993) In: Chromatography in Biotechnology: ACS Symposium Series 529 (Horva'th, C. and Ettre, L. S., Eds.) American Chemical Society, Washington, D.C.; Scott (1992) In: Food Analysis by HPLC (L. M. L. Nollet, Ed.), Marcel Dekker, Inc., New York, N.Y.); and Lee, Anal. Biochem. 179, 404-412, 1990).

For example, normal phase HPLC using amine-bonded silica matrices is useful for resolving underivatized sugars and radiolabeled alditols (Mellis and Baenziger, Anal. Biochem. 114, 276-280, 1981). Reverse-phase methods, using ODS-silica are useful for resolving derivatized sugars (Tomiya et al., Anal. Biochem. 163, 489-499, 1987). Anion-exchange methods, such as, for example, using DEAE (Pharmacia) or Mono-Q (Pharmacia), are useful for resolving sialylated, phosphorylated, or sulfated sugars (Watson and Bhide, Liq. Chrom/Gas Chrom. 11, 216-220, 1993). High Pressure Anion Exchange Chromatography methods, using a strong anion exchanger at high pH (e.g. Dionex or CarboPac) are also useful in this respect (Townsend and Hardy, Glycobiol. 1, 139-147, 1991).

Serial Lectin Affinity chromatography, using a range of immobilized lectin ligands, particularly in combination with HPLAC, is useful for resolving a number of sugars, such as galactose, fucose, N-acetyl glucosamine (GlcNAc), mannose, glucose, or N-acetyl galactosamine (GalNAc) (see Cummings et al., Methods Cell Biol. 32, 141-183, 1989; and Virgilio (1998) In: Lectins, Biology, Biochemistry, Clinical Biochemistry, Vol. 12, including Proceedings from the 17^{th} Int. Lectin Meeting, Wurzburg, 1997 (van Driessche, E., Beeckmans, S., and Bog-Hansen, T., eds), Textop publishers, Hellerup, Denmark (ISBN 87-984583-0-2). Exemplary lectins include Canavalia ensiformis concanavalin A (ConA), galectin-I, Phytolacca americana pokeweed mitogen (PWM), P. americana Pa-2, and any one or more of the agglutinins from Agaricus bisporus (ABA-I), Aleuria aurantia (AAA), Allomyrina dichotoma (Allo A-I/II), Arachis hypogea (PNA), Bauhinia purpurea (BPA), Datura stramonium (DSA), Dolichos biflorus (DBA), Erythrina cristagalli (EclA), Erythrina corallodendron (EcoA), Erythrinia variegata (EVA), Galanthus nivalis (GNA), Griffonia simplicifolia (I A4 or GSA-A4; I B4 or GSA-B4; II or GSA-II), Lens culinaris (LCA),Lotus tetragonolobus (LTA), Lycopersicon esculentum (LEA), Maakia amurensis (MAA), Oryza sativa (OSA), Phaseolus vulgaris (erythroagglutinin or E-PHA; leukoagglutinin or L-PHA), Pisum sativum (PSA), Ricinus communis (RCA-I, RCA-II), Sambucus nigra (SNA), Sophora japonica (SJA), Triticum vulgaris wheat germ (WGA), Ulex europeaus (UEA-I), Vicia faba (VFA), Vicia graminea (VGA), Vicia villosa (VVA-B4), or Wisteria floribunda (WFA).

Alternatively, or in addition, High Pressure Anion Exchange Chromatography methods, such as, for example HPAEC/PAD can be used to separate complex carbohydrate-containing and non-carbohydrate-containing mixtures, particularly the anionic molecule of the invention, or a phosphate-containing or sulfate-containing fragment thereof.

Alternatively, or in addition, size exclusion chromatography (Kobata et al., Methods Enzymol. 138, 84-94, 1987; Oxford GlycoSystem's GlycoMap 1000) is used for the resolution of the fragments, the separation being based upon their size.

The present inventors have also shown reverse-phase HPLC (RP-HPLC) to be useful to separate the cancer marker of the invention from other, more hydrophobic molecules, such as, for example, hydrophobic gangliosides and ceramides. This is because the molecule of the present invention is hydrophilic. Notwithstanding that this is the case, RP-HPLC is useful for the resolution of fragments of the carbohydrate or other moiety, particularly if they are chemically derivatized to introduce a hydrophobic chromophore or fluorophore, such as, for example, by reductive amination using 2-aminopyridine. As an example, sugars have been labeled using 2-aminopyridine, and are amenable to mapping, essentially as described by Tomiya etal., Anal. Biochem. 171, 73-90, 1988.

Electrophoretic methods, such as, for example, paper electrophoresis, capillary electrophoresis, and preferably, gel electrophoresis using high-percentage polyacrylamide slab gels, can be used to separate fluorescent derivatives of the fragments (e.g. Fluorophore Assisted Carbohydrate Electrophoresis (FACE), Millipore).

The use of mass spectrometry (MS), or tandem MS (e.g. MS/MS, MALDI-TOF/electrospray MS, electrospray MS/MALDI-TOF, MALDI-TOF/post-source MALDI-TOF, etc) is particularly preferred for resolving carbohydrate-containing and non-carbohydrate-containing fragments, especially when combined with NMR, chemical, or exoglycosidase degradation, to determine the identity, linkage positions, and anomericity of carbohydrate-containing and non-earbohydrate-containing fragments, including any resolved monosaccharides, disaccharides, or oligosaccharides. Those skilled in the art will be aware that mass spectrometry is an analytical technique for the accurate determination of molecular weights, the identification of chemical structures, the determination of the composition of mixtures, and qualitative elemental analysis. In operation, a mass spectrometer generates ions of sample molecules under investigation, separates the ions according to their mass-to-charge ratio, and measures the relative abundance of each ion. Preferably, the mass spectrometry system used MALDI-TOF MS or electrospray MS or a post-source fragmentation method thereof. The general steps in performing a mass-spectrometric analysis are as follows:

(i) create gas-phase ions from a sample;

(ii) separate the ions in space or time based on their mass-to-charge ratio; and

(iii) measure the quantity of ions of each selected mass-to-charge ratio.

Time-of-flight (TOF) mass spectrometers, such as, for example, those described in U.S. Pat. Nos. 5,045,694 and 5,160,840, generate ions of sample material under investigation and separate those ions according to their mass-to-charge ratio by measuring the time it takes generated ions to travel to a detector. TOF mass spectrometers are advantageous because they are relatively simple, inexpensive instruments with virtually unlimited mass-to-charge ratio range. TOF mass spectrometers have potentially higher sensitivity than scanning instruments because they can record all the ions generated from each ionization event. TOF mass spectrometers are particularly useful for measuring the mass-to-charge ratio of large organic molecules where conventional magnetic field mass spectrometers lack sensitivity. The flight time of an ion accelerated by a given electric potential is proportional to its mass-to-charge ratio. Thus the time-of-flight of an ion is a function of its mass-to-charge ratio, and is approximately proportional to the square root of the mass-to-charge ratio. Assuming the presence of only singly charged ions, the lightest ion reaches the detector first, followed by successively heavier mass groups. TOF mass spectrometers thus provide an extremely accurate estimate of the mass/charge ratio of a molecular species under investigation, and the error, generally no more than m/z±5, is largely a consequence of ions of equal mass and charge not arriving at the detector at exactly the same time. This error occurs primarily because of the initial temporal, spatial, and kinetic energy distributions of generated ions that lead to broadening of the mass spectral peaks, thereby limiting the resolving power of TOF spectrometers. The initial temporal distribution results from the uncertainty in the time of ion formation. The certainty of time of ion formation is enhanced by pulsed ionization techniques, such as, for example, plasma desorption and laser desorption, that generate ions during a very short period of time and result in the smallest initial spatial distributions, because ions originate from well defined areas on the sample surface and the initial spatial uncertainty of ion formation is negligible. Pulsed ionization such as plasma desorption (PD) ionization and laser desorption (LD) ionization generate ions with minimal uncertainty in space and time, but relatively broad initial energy distributions. Because long pulse lengths can seriously limit mass resolution, conventional LD typically employs sufficiently short pulses (frequently less than 10 nanoseconds) to minimize temporal uncertainty. The performance of LD is enhanced by the addition of a small organic matrix molecule to the sample material, that is highly absorbing, at the wavelength of the laser (i.e. Matrix-assisted laser desorption/ionization, hereinafter “MALDI”). The matrix facilitates desorption and ionization of the sample. MALDI is particularly advantageous in biological applications since it facilitates desorption and ionization of large biomolecules in excess of 100,000 Da molecular mass without their fractionation. A preferred matrix for performing the instant invention comprises 2-(4-hydroxyphenylazo) benzoic acid (HABA), also known as 4-hydroxybenzene-2-carboxylic acid. In MALDI, samples are usually deposited on a smooth metal surface and desorbed into the gas phase as the result of a pulsed laser beam impinging on the surface of the sample. Thus, ions are produced in a short time interval, corresponding approximately to the duration of the laser pulse, and in a very small spatial region corresponding to that portion of the solid matrix and sample which absorbs sufficient energy from the laser to be vaporized. MALDI provides a near-ideal source of ions for time-of-flight (TOF) mass spectrometry, particularly where the initial ion velocities are small. Considerable improvements in mass resolution are obtained using pulsed ion extraction in a MALDI ion source. Ion reflectors (also called ion mirrors and reflectrons, consisting of one or more homogeneous, retarding, electrostatic fields) are also known to compensate for the effects of the initial kinetic energy distribution of the analyte ions, particularly when positioned at the end of the free-flight region. Additional improvements to MALDI are known in the art with respect to the production of ions from surfaces, by improving resolution, increasing mass accuracy, increasing signal intensity, and reducing background noise, such as, for example, those improvements described in U.S. Pat. No. 6,057,543.

Electrospray MS, or electrospray ionization MS, is used to produce gas-phase ions from a liquid sample matrix, to permit introduction of the sample into a mass spectrometer. Electrospray MS is therefor useful for providing an interface between a liquid chromatograph and a mass spectrometer. In electrospray MS, a liquid analyte is pumped through a capillary tube (hereinafter “needle”), and a potential difference (e.g. three to four thousand Volts) is established between the tip of the needle and an opposing wall, capillary entrance, or similar structure. The stream of liquid issuing from the needle tip is diffused into highly-charged droplets by the electric field, forming the electrospray. An inert drying gas, such as, for example, dry nitrogen gas, may also be introduced through a surrounding capillary to enhance nebulization of the fluid stream. The electrospray droplets are transported in an electric field and injected into the mass spectrometer, which is maintained at a high vacuum. Through the combined effects of a drying gas and vacuum, the carrier liquid in the droplets evaporates gradually, giving rise to smaller, increasingly unstable droplets from which surface ions are liberated into the vacuum for analysis. The desolvated ions pass through sample cone and skimmer lenses, and after focusing by a RF lens, into the high vacuum region of the mass-spectrometer, where they are separated according to mass and detected by an appropriate detector (e.g., a photo-multiplier tube). Preferred liquid flow rates of 20-30 microliters/min are used, depending on the solvent composition. Higher liquid flow rates may result in unstable and inefficient ionization of the dissolved sample, in which case a pneumatically-assisted electrospray needle may be used.

Sample preparation for introduction into the MS environment generally involves desalting, essentially as described in Example 1, preferably an additional fractionation, such as, for example, using reverse phase, prior to analysis using at least one standard chromatographic separation or purification step. Derivatization of the fragments to enhance their surface activity, such as, for example, by sequential periodate oxidation, NaBD₄ reduction, and permethylation (Nilsson, 1993, In: Glycoprotein Analysis in Biomedicine (E. F. Hounsell, Ed.) Humana Press, Totowa, N.J., pp 35-46) or derivatization with perfluorobenzylaminobenzoate or reducing-terminal modification with alkyl-aminobenzoates, can improve sensitivity and/or resolving power of the method. In cases where MALDI-TOF MS is employed, the sample will be mixed with a suitable matrix and dried, whereas in the case of electrospray MS, the sample will be injected directly as a liquid sample in an appropriate carrier solution.

Furthermore, a derivative of the cancer marker described herein or a fragment thereof shall also be taken to include any molecules produced by the addition of one or more fluorescent ligands, chromophores, enzyme ligands, radioactive ligands, peptide ligands (e.g. FLAG), or antibody ligands, to a carbohydrate or other moiety of said molecule. Procedures for the addition of such ligands to carbohydrates and other moieties are well known in the art.

For additional reviews of methods for analyzing carbohydrates and glycopolymers, and the types of derivatives that can be produced therefrom, see Hounsell, Adv. Carbohydr. Chem. Biochem., 50, 311-350, 1994; Hounsell, (1997) In: Glycoscience: Status and Perspectives (H. J. Gabius and S. Gabius, eds), Chapman and Hall. pp 15-29; and Hounsell (1997) Editor In: Glycoscience Protocols Methods in Molecular Biology, Humana Press.

While not being bound by any theory or mode of action, it is possible that the molecule of the invention is immune system dependent in so far as it requires the presence of an activated or functional immune system for its expression, and/or is secreted into the circulation and other bodily fluids in healthy subjects. Accordingly, tumorigenesis may reduce its expression and/or secretion and/or cause its shedding from cells on which it is normally produced during tumorigenesis, such as before metastases.

The determination of this m/z 991 ion cancer marker by the present inventors, in particular the elucidation of its expression profile in both normal and cancer cells, and the provision of an assay system for its detection, facilitates a range of methods for the diagnosis of cancer in both human and non-human mammalian subjects.

Accordingly, in another aspect of the present invention provides a method of diagnosing or detecting cancer in a human or non-human mammalian subject comprising: (i) determining the level of a cancer marker in a test sample from a subject suspected of having cancer, said cancer marker comprising a negatively-charged molecule having a m/z ratio of about 991 or a derivative thereof; and

(ii) comparing the level of the cancer marker or derivative at (i) to the level of the cancer marker or derivative in a control sample from a healthy subject, wherein a reduced level of said cancer marker or derivative relative to the level in the healthy subject is indicative of cancer.

However, a control sample need not be used if a control, healthy subject, range has been established previously so that measurements made in the test sample can be compared to the control range. Also, an internal sample control may be used to assess the degree of reduction in the level of the cancer marker. For example, another molecule (ie. another marker) within the test sample, which shows stable levels in both test and control, samples, may be chosen to calculate a ratio, wherein a change in the ratio of the cancer marker to the another marker is indicative of cancer. Alternatively, the test sample may be “spiked” with a suitable standard marker, thus providing an internal standard. A number of such markers are available or can be easily derived by those skilled in the art of mass spectrometry.

Any art-recognized method, such as, for example, immune detection, chromatography (hydrophobic interaction chromatography, high pressure liquid chromatography, reverse phase chromatography, or lectin affinity chromatography, amongst others) can be employed to assay the level of the cancer marker in the subject relative to the level in a healthy subject. Preferably, albeit not necessarily, mass spectrometry is employed in the diagnosis. These processes for detecting or measuring the molecule of the invention or a fragment thereof are broadly described herein above.

The present invention is particularly directed to the diagnosis of a cancer of neuroectodennal origin, preferably a cancer selected from the group consisting of carcinoma, lymphoma, and sarcoma, such as, for example, ovarian cancer, colon cancer, breast cancer, pancreatic cancer, lung cancer, prostate cancer, urinary tract cancer, uterine cancer, acute lymphatic leukemia, Hodgkin's disease, melanoma, neuroblastoma, glioma, and soft tissue sarcoma. In a particularly preferred embodiment of the invention the cancer is selected from the group consisting of: melanoma, adenocarcinoma, and colon cancer.

It will be apparent that the diagnostic method described herein is not limited to the diagnosis of cancer, but can be applied to monitoring the progress of the disease in a particular subject, by comparing the level of the cancer marker in the subject over time. In the case of a patient in remission, a sample taken early in remission can be used as a standard for comparison against later samples. Preferably from the same bodily fluid as the earlier sample, to determine the status of the subject, since any further modification to the level of a cancer marker may indicate that the period of remission has ended. Similarly, for a patient who has undergone treatment successfully leading to a remission or cure, or who has not exhibited any metastases, a sample taken shortly after treatment or prior to metastases can be used as a standard for comparison against later samples, to determine whether or not the subject has suffered recurrence or metastases of the tumor, since any modified level of a cancer marker may indicate recurrence or metastases.

The term “subject suspected of having cancer” will be understood to mean that the subject has exhibited one or more symptoms associated with a cancer, or has previously been diagnosed as having cancer at the time of obtaining the test sample used as a test sample in the inventive method, including a subject in remission from cancer wherein the remission period is suspected of drawing to a close or is being monitored.

As used herein, the term “healthy subject” shall be taken to mean a subject that has not exhibited any symptoms associated with cancer when the control sample was taken, or is in remission from the symptoms associated with cancer when the control sample was taken, or has not exhibited any metastases of a previously-diagnosed tumor in the blood or serum, or other bodily fluids, at the time when the blood fraction was taken. Accordingly, the “healthy subject” need not be distinct from the subject suspected of having cancer. For example, a particular individual, such as, for example an individual at risk of developing cancer, may provide bodily fluid samples at different times, in which case an early sample taken prior to any symptom development may be used as a control sample against a later sample being tested. Alternatively, a bodily fluid sample taken from a subject in remission, or following treatment, may be used as a control sample against a sample from the same subject taken earlier or later, such as, for example, to monitor the progress of the disease.

By “control sample” is meant a sample having a known composition or content of a particular integer against which a comparison to a test sample is made. The only requirement for the source of a control sample is that it does not contain a level of the cancer marker being detected that is consistent with the disease state.

The test sample or control sample used in the assay described herein can be any bodily fluid sample from the subject suspected of having a cancer or the healthy subject, such as, for example, a blood fraction, serum fraction, urine, saliva, mucus, sputum, or tears, amongst others. In a particularly preferred embodiment, the control sample or the test sample is a blood fraction, preferably a serum fraction.

As used herein, a “blood fraction” means any derivative of blood, and shall be taken to include a supernatant or precipitate of blood, a serum fraction or plasma fraction, a buffy coat fraction, a fraction enriched for T-cells, a fraction enriched for platelets, a fraction enriched for platelets erythrocytes, a fraction enriched for basophils, a fraction enriched for eosinophils, a fraction enriched for lymphocytes, a fraction enriched for monocytes, a fraction enriched for neutrophils, or any partially-purified or purified component or blood whether or not in admixture with any other component of blood. Blood fractions may be obtained, for example, by treatment of blood with a precipitant (e.g. low temperature, acid, base, ammonium sulfate, polyethylene glycol, etc), or fractionation by chromatography (e.g. size exclusion, ion exchange, hydrophobic interaction, reverse phase, mass spectrometry, etc).

In the present context, the term “serum fraction” means a sample derived from serum. Exemplary serum fractions include a plasma protein fraction (e.g. albumin fraction, fibrinogen (factor I) fraction, serum globulin fraction, factor V fraction, factor VIII fraction, or prothrombin complex fraction comprising factors VII, IX and X), a cryosupematant or cryoprecipitate of plasma, a cryosupematant or cryoprecipitate of fresh frozen plasma, a cryosupernatant or cryoprecipitate of a plasma fraction, or any partially-purified or purified component of serum whether or not in admixture with any other serum component. Serum fractions may be obtained, for example, by treatment of serum with a precipitant (e.g. low temperature, acid, base, ammonium sulfate, polyethylene glycol, etc), or by fractionation using chromatography (e.g. size exclusion, ion exchange, hydrophobic interaction, reverse phase, mass spectrometry, etc).

Because the method of the present invention is performed on bodily fluid samples, it is convenient to perform and non-invasive.

Depending upon the analytical technique used, bodily fluid samples are prepared by standard methods known to those skilled in the art or prepared according to the methods described herein without undue experimentation. The present invention clearly encompasses the preparation and handling of samples subjected to the diagnostic assay described herein.

By “comparing the level of the cancer marker or derivative at (i) to the level of the cancer marker or derivative in a control sample from a healthy subject” is meant that the amount or concentration of the cancer marker or derivative of the inventive molecule is compared between the control sample and the test sample. This is readily performed, for example, where mass spectrometry is used to analyze the relative amounts of cancer marker in the two samples as a percentage of the most abundant peak. For example, conditions for mass spectrometry of a sample can be manipulated to ensure that the peak height of a particular molecular species, or the area of a particular peak, is proportional to the abundance of that molecular species in the sample. Accordingly, it is not strictly necessary to conduct a further assay of a collected peak sample to determine the abundance of the molecular species therein, because the spectra of two samples may be overlaid to determine the differences in peak heights. Alternatively, or in addition to determining the relative level of the cancer marker, it is possible to determine the absolute concentration of the cancer marker by integration of the peak heights, or by further biochemical assay or immune assay of the peak corresponding to the cancer marker. However, for quantitation, it is preferred that only a crude sample preparation is performed.

The present invention clearly includes the step of determining the abundance of the cancer marker of the invention in either the test sample or control sample, and/or the relative abundance of the cancer marker in said samples. This includes determining the abundance or relative abundance of the cancer marker in the blood or serum from which any blood fraction or serum fraction is derived. Standard assays may be employed for this purpose, such as, for example, an immunochemical analysis of the peak fraction.

Preferably, this aspect of the invention further includes the first step of obtaining the bodily fluid sample, or any intermediate fraction derived therefrom (e.g. a precipitate of a crude mixture of glycan, glycolipid and carbohydrate).

Preferably, the method according to this aspect of the invention includes the further characterization of the cancer marker or derivative, in particular according to its mass/charge ratio and/or molecular mass and/or structure, to confirm its identity. As will be apparent from the preceding discussion, these properties are readily determined using art recognized procedures. In a particularly preferred embodiment, the mass/charge ratio of the molecule of the invention, or the mass/charge ratio of one or more of its post-source ionization fragments, or the profile of post-source ionization fragments, is determined to confirm the identity of the cancer marker, such as, for example, by mass spectrometry against calibrated markers, with a maximum error in the estimated mass/charge ratio of ±5, more preferably ±4, even more preferably ±3, still more preferably ±2, and even still more preferably ±1.

For the immunological assay of the cancer marker of the invention, monoclonal antibodies are prepared against the cancer marker, preferably against a purified molecule or derivative thereof, such as, for example, a fraction from mass spectrometry, and then used in standard immunoassay techniques for the subsequent diagnosis of cancer.

To prepare the monoclonal antibodies, mice or other mammals can be pretreated by injection with low doses of cyclophosphamide (15 mg/Kg non-human mammalian body weight) to reduce their suppressor cell activity, and then immunized with various doses of the molecule, at short intervals (i.e. between 3-4 days and one week). By virtue of the glycophosphoinositol moiety, the molecule can be introduced into a liposome, which is subsequently used for immunizing the animals, essentially as described in U.S. Pat. No. 5,817,513. Immunizations are performed by subcutaneous, intravenous, or intraperitoneal injection, in accordance with standard procedures. Before and during the immunization period, blood serum samples are taken from the animals for monitoring antibody titers generated against the molecule used as an antigen, by any known immunoassay method for detecting an antigen-antibody reaction. In general, about 5-9 accumulative doses of a liposome preparation at short time intervals will facilitate an antibody response to the molecule. Mice with serum antibody titers against the molecule receive a new immunization with the liposome preparations, about three days before obtaining antibody producing cells, and then the antibody producing cells, preferably spleen cells, are isolated. These cells are fused with myeloma cells to produce hybridomas in accordance with standard procedures for preparing monoclonal antibodies. The titres of the monoclonal antibodies produced by the hybridomas are then tested by immunoassay methods.

Preferably, an immuno-enzymatic assay is employed, in which hybridoma supernatants bind to a test sample containing the antigen and then antigen-antibody binding is detected using a second enzyme labelled antibody that binds to the monoclonal antibody. Once the desired hybridoma is selected and sub-cloned, such as, for example, by limiting dilution, the resulting monoclonal antibody can be amplified in vitro in an adequate medium, during an appropriate period, followed by the recovery of the desired antibody from the supernatant. The selected medium and the adequate culture time period are known to the skilled person, or easily determined.

Another production method comprises the injection of the hybridoma into syngeneic mice. Under these conditions, the hybridoma causes the formation of non-solid tumors, which will produce a high concentration of the desired antibody in the blood stream and the peritoneal exudate (ascites) of the mice.

Standard immunoassays are then used to assay for the presence of the molecule in a test sample and/or control sample.

A third aspect of the invention clearly contemplates a monoclonal antibody that is cross-reactive with the molecule of the present invention, or a carbohydrate moiety, lipid moiety, or protein moiety thereof.

A fourth aspect of the invention contemplates a diagnostic kit for the detection of cancer in a human or other mammalian subject, said kit comprising an amount of the isolated molecule of the invention suitable for use as a calibration standard and one or more buffers suitable for use.

By “calibration standard” is meant that a reference sample for assisting in determining the amount of a stated integer and/or one or more physical properties of said integer. Generally the calibration standard is in isolated form to minimize spurious results arising from contaminants. Accordingly, a control sample of the diagnostic assay described herein may be a calibration standard.

The buffer will be any buffer suitable for suspending the calibration standard or control sample, and/or the test sample for subsequent assay using immunological means, mass spectrometry, or other detection means. Alternatively, or in addition, the buffer may be any buffer suitable for conducting the antibody-antigen binding reaction during immune detection assay of the molecule of the invention.

In an alternative embodiment, the invention contemplates a diagnostic kit for the detection of cancer in a human or other mammalian subject, said kit comprising an amount of an antibody that binds specifically to the isolated molecule and one or more buffers suitable for use.

Preferably, the antibody is a monoclonal antibody.

In a further alternative embodiment, this invention contemplates a diagnostic kit for the detection of cancer in a human or other mammalian subject, said kit comprising an amount of the isolated molecule of the invention suitable for use as a calibration standard, an antibody that binds specifically to the isolated molecule, and one or more buffers suitable for use.

The kit according to any one or more of the preceding embodiments is preferably supplied with instructions for use. The use of these kits will be understood by those skilled in the art, based upon the description provided herein.

The non-limiting examples presented below are intended to further describe the isolated molecule of the present invention and its use in detecting a range of different cancers in humans and other mammals.

### EXAMPLES

### Example 1

Loss of an m/z 991 Ion from the Blood of Tumor Bearing Animals and Humans

A. Materials and Methods

1. Tumor Models

Rats: Rats were female Fischer 344 rats carrying the highly metastatic rat mammary adenocarcinoma 13762 MAT (Parish et al., Int. J. Cancer 40, 511-518, 1987). Tumor cells were maintained in vitro as previously described (Parish et al., Int. J. Cancer 40, 511-518, 1987). To induce tumors in rats, the animals (10-13 weeks of age) were injected s/c with 10⁶ 13762 MAT cells and tumors (15-17 mm diameter) appeared about 13 days later.

Mice: The highly malignant and metastatic B16F1 melanoma cell line was injected s/c (10⁶ cells/mouse) into female C57BL/6 mice, and tumors (12-14 mm diameter) appeared about 15 days later.

Humans: Subjects diagnosed with colon cancer were used, and citrated plasma was collected therefrom.

2. Serum and Plasma Samples

Blood was collected with or without anticoagulant (citrate-phosphatedextrose) from healthy human subjects and subjects having colon cancer, or alternatively, from healthy and tumor-bearing C57BL/6 mice or healthy or tumor-bearing Fischer 344 rats. Following collection, non-anticoagulated blood was incubated at 37° C. for 30 min, stored at 4° C. overnight, and then sera collected. Plasma samples were obtained following centrifugation (4000×g, 12 min) of the anticoagulated blood.

3. Fractionation of Serum—Ammonium Sulfate/pyridine Method

Serum or plasma (2-3 ml) was acidified (pH 5.5 to pH 5.8) with hydrochloric acid (HCl). Some of the protein was precipitated out by mixing the serum for 3 h at 4° C. with one volume of supersaturated ammonium sulfate. The mixture was spun at 10,000×g for 10 min at 4° C. and the supernatant collected. Further deproteination was performed by adding powdered ammonium sulfate to give 90-95% saturation, followed by mixing overnight at 4° C. The mixture was spun at 100,000×g for 1 hour at 4° C. and the supernatant collected. Acetonitrile (four volumes) was then added to the supernatant while stirring continuously at 4° C. The mixture was left to stand for 5 min before the acetonitrile layer was decanted and collected. The rest of the mixture was spun at 1500×g for 5 min and the remaining acetonitrile layer collected. The acetonitrile fractions were combined and the solvent evaporated off. The residue was resuspended in chloroform/methanol/water (CMW; 2/43/55; 1 ml) and applied twice onto a pre-equilibrated C₁₈ Seppak cartridge (Waters, Taunton, Me.). The eluate (unadsorbed fraction) was collected. The vessel was washed with CMW (1 ml) and the wash passed through the cartridge. The eluate was collected with the unadsorbed fraction. The cartridge was then sequentially eluted with 2 ml each of water, methanol/water, methanol, chloroform/methanol and chloroform. All fractions were collected separately. The fractions were dried under vacuum (SpeedVac). The unadsorbed fraction and the water fraction were resuspended in the minimum amount of water and dialysed extensively against water using a 1 kDa molecular weight cut off dialysis membrane. The dialysates were dried under vacuum (SpeedVac). The fractions were redissolved in 10 μl of the relevant solvent and analysed by MALDI-TOF MS as described below.

4. MALDI-TOF MS Analysis

To prepare samples for mass spectrometry, the fractions were dried in vacuo. The flow through fraction and the methanol/water fraction were dissolved in water (200 μl), dialyzed extensively against water using a 1 kDa molecular weight cut off dialysis membrane, and dried by evaporation. All fractions were re-dissolved in 10 μl of the relevant solvent for loading into the mass spectrometer.

Fractions prepared as described supra (1 μl) and mixed, by vortex, with matrix solution [2 μl of a 3.5 mg/ml solution of 2-(4-hydroxyphenylazo) benzoic acid (HABA) in methanol]. The mixture (1 μl) was loaded onto a sample plate having 96 loading positions, and dried at room temperature. The sample plate was then loaded into the MALDI-TOF MS (TofSpec-2e; Micromass, Manchester, UK or Voyager Elite-DE; BioPerceptive). A nitrogen laser (337 nm) was used for ionization, and the analysis was carried out in the linear or reflector negative ion mode. Post source decay (PSD) fragmentation was performed on some samples containing the ion of interest. Data are presented as m/z ratio profiles showing the mass charge ratio of each peak, with peak heights being depicted as the percentage height of the most abundant molecular species detected in the sample.

Results

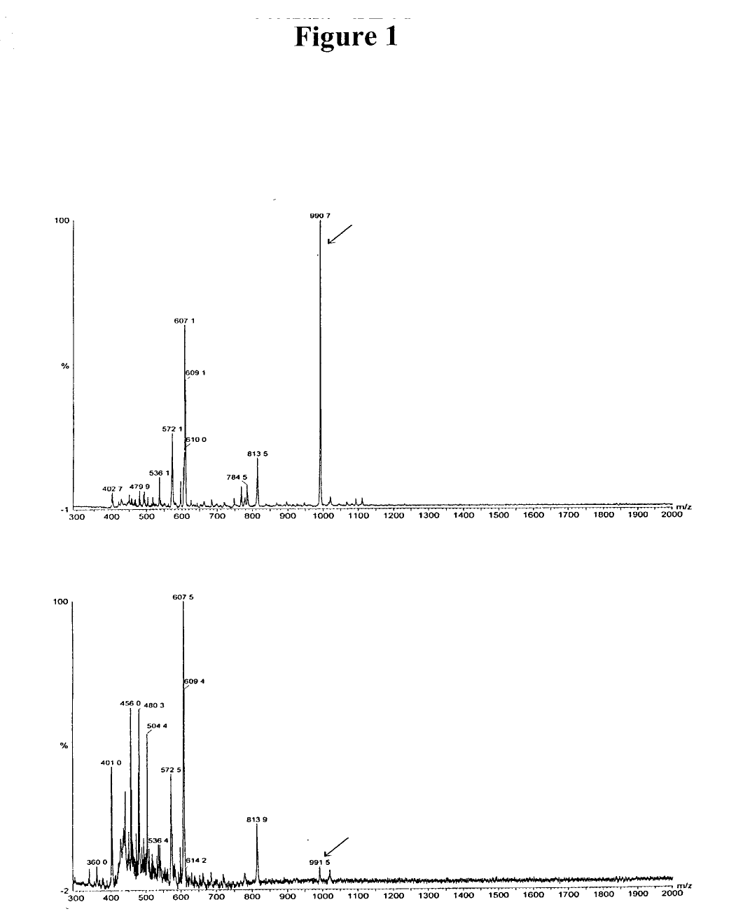

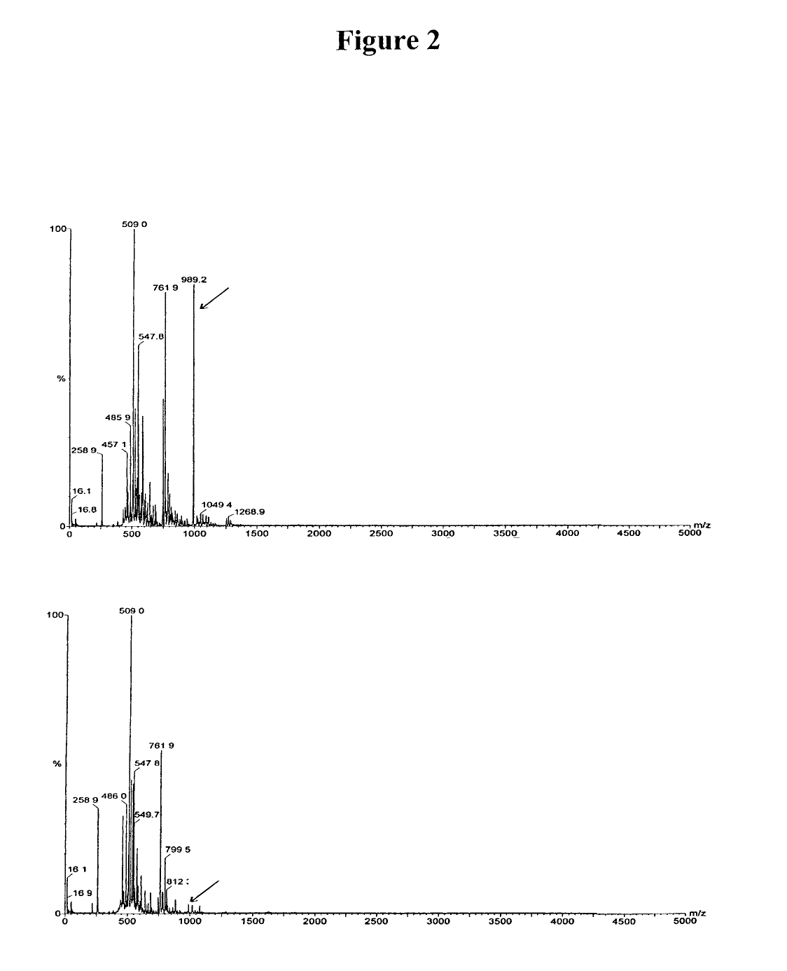

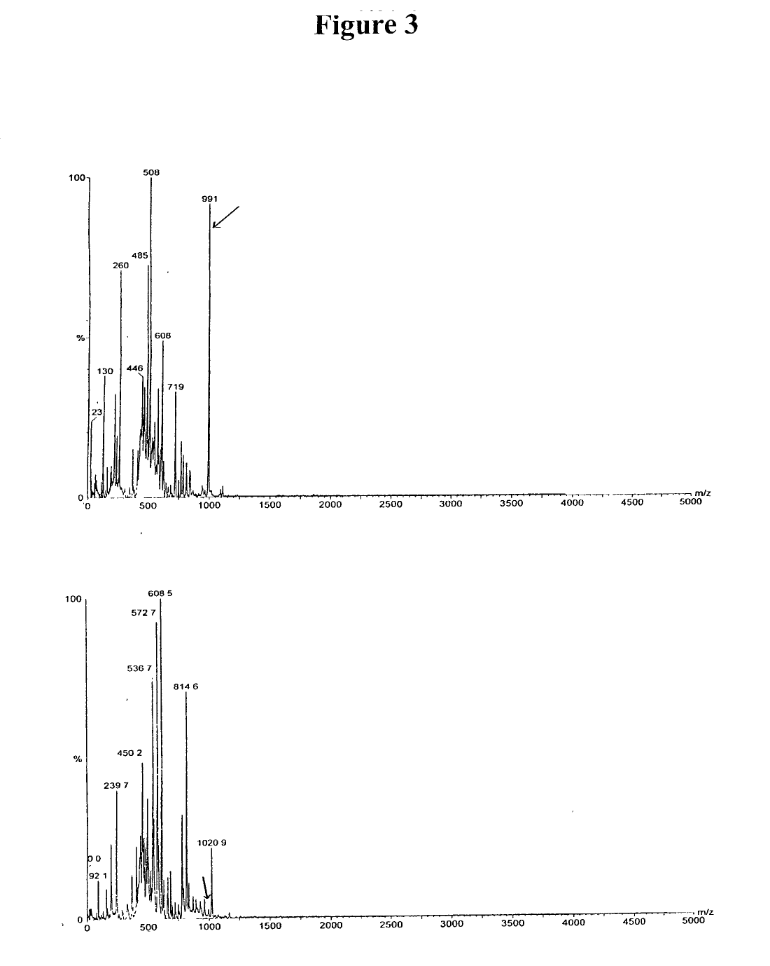

We found that the flow through fraction (i.e. the fraction that did not adsorb to the C₁₈ Seppak column) from the sera of healthy rats, mice or humans contained a very prominent negative ion species having a m/z ratio of about 991, when analyzed by MALDI-TOF MS (FIGS. 1A, 2A, and 3A). This negative ion was absent from the sera of tumor bearing rats (FIG. 1B), tumor-bearing mice (FIG. 2B), and the plasma of colon cancer patients (FIG. 3B).

Post source decay fragmentation of the m/z 991 ion was essentially identical in all of the three species tested (FIG. 4A, FIG. 4B, and FIG. 4C), suggesting that the molecule is identical in rats, mice and humans.

Additional studies revealed that the ion of m/z 991 was absent from the sera of mice only 2 days after subcutaneous injection of 10⁶ B16 melanoma cells. At this time there was no palpable tumor present in the mice which further indicates the potential for using this cancer marker in the early diagnosis of cancer.

Although the present invention has been described with reference to particular preferred embodiments and examples, it will be clear to those skilled in the art that variations and modifications of the invention, in keeping with the general principles and spirit of the invention, are also encompassed herein.

What is claimed is:

1. An isolated or purified cancer marker comprising a negatively-charged molecule or a derivative thereof, wherein said molecule has an m/z ratio of about 991, and wherein said molecule is present at a reduced level in a subject having a cancer compared to a healthy subject.

2. The cancer marker of claim 1 wherein the marker is a derivative that comprises at least one fragment of the negatively-charged molecule.

3. The cancer marker of claim 2 wherein said at least one fragment has an m/z ratio selected from the group consisting of about 241, about 644, about 705, about 749, and about 947.

4. The cancer marker of claim 3 comprising at least two of said fragments.

5. The cancer marker of claim 3 comprising at least three of said fragments.

6. The cancer marker of claim 3 comprising at least four of said fragments.

7. The cancer marker of claim 3 comprising all five of said fragments.

8. The cancer marker of claim 1 wherein the marker is a derivative that comprises the negatively-charged molecule covalently attached to a ligand selected from the group consisting of: a fluorescent ligand, an enzyme ligand, a radioactive ligand, a peptide ligand, and an antibody ligand.

9. An isolated or purified cancer marker comprising a negatively-charged molecule, wherein said molecule has an m/z ratio of about 991, wherein said molecule is present at a reduced level in a subject having a cancer compared to a healthy subject, and wherein said molecule is comprised of five fragments having an m/z ratio of about 241, about 644, about 705, about 749, and about 947.

10. A method of diagnosing or detecting cancer in a human or non-human mammalian subject comprising:
(i) determining the level of a cancer marker in a test sample from a subject suspected of having cancer, wherein said cancer marker comprises a negatively-charged molecule having an m/z ratio of about 991 or a derivative thereof; and
(ii) comparing the level of the cancer marker or derivative at (i) to the level of the cancer marker or derivative in a control sample from a healthy subject, or the level established for a healthy subject, wherein a reduced level of said cancer marker or derivative relative to the level in the healthy subject, or the level established for a healthy subject, is indicative of cancer.

11. The method of claim 10 wherein the level of the cancer marker is determined by mass spectrometry or chromatography techniques.

12. The method of claim 10 wherein the cancer is of neuroectodermal origin.

13. The method of claim 10 wherein the cancer is selected from the group consisting of carcinoma, lymphoma, and sarcoma.

14. The method of claim 10 wherein the cancer is a melanoma.

15. The method of claim 10 wherein the cancer is adenocarcinoma.

16. The method of claim 10 wherein the cancer is a colon cancer.

17. The method of claim 10 wherein the test sample and/or the control sample is a bodily fluid or a fraction thereof.

18. The method of claim 17 wherein the test sample is blood or a fraction thereof.

19. The method of claim 18 wherein the test sample is serum or a derivative fraction thereof.

20. The method of claim 10, further comprising determining the abundance of the cancer marker in either the test sample or control sample, and/or the relative abundance of the cancer marker in said samples.

21. The method of claim 10, further comprising a first step of obtaining the sample.

22. The method of claim 10, further comprising confirming the identity of the cancer marker by determining its fragmentation profile.

23. A method of diagnosing or detecting cancer in a human or non-human mammalian subject comprising:
(i) determining the level of a cancer marker in a test sample from a subject suspected of having cancer, wherein said cancer marker comprises a negatively-charged molecule having a m/z ratio of about 991 or a derivative thereof; and
(ii) comparing the level of the cancer marker or derivative at (i) to the level of an internal standard added to the test sample, wherein a reduced level of said cancer marker or derivative relative to the level of the internal standard is indicative of cancer.

24. A method of diagnosing or detecting cancer in a human or non-human mammalian subject comprising:
determining the level of a cancer marker in a test sample from a subject suspected of having cancer, wherein said cancer marker comprises a negatively-charged molecule having an m/z ratio of about 991 or a derivative thereof;
determining the level of a second marker in the same test sample; and
comparing the levels of the two markers, wherein a change in the ratio of the cancer marker to the second marker is indicative of cancer.

25. A method of monitoring cancer treatment in a human or non-human mammalian subject comprising:
(i) determining the level of a cancer marker in a test sample from a subject being treated for cancer, wherein said cancer marker comprises a negatively-charged molecule having an m/z ratio of about 991 or a derivative thereof; and
(ii) comparing the level of the cancer marker or derivative at (i) to the level of the cancer marker or derivative in a control sample from a healthy subject, or the level established for a healthy subject, wherein an increased level is indicative of successful treatment.

26. A method of diagnosing recurrence of cancer following successful treatment in a human or non-human mammalian subject comprising:
(i) determining the level of a cancer marker in a test sample from a subject treated for cancer, wherein said cancer marker comprises a negatively-charged molecule having an m/z ratio of about 991 or a derivative thereof; and
(ii) comparing the level of the cancer marker or derivative at (i) to the level of the cancer marker or derivative in a control sample from a healthy subject, the level established for a healthy subject or the level in a sample from the subject following cancer treatment, wherein a reduced level is indicative of recurrence of cancer.
